(19) United States
(12) Patent Application Publication
(10) Pub. No.: US 2026/0021027 A1
(43) Pub. Date: Jan. 22, 2026

(54) POLYPEPTIDE COSMETIC COMPOSITIONS

(71) Applicant: ELC Management LLC, Melville, NY (US)

(72) Inventors: Jacqueline TRIVERO, Putnam Valley, NY (US); Nadine PERNODET, Dix Hills, NY (US)

(73) Assignee: ELC Management LLC, Melville, NY (US)

(21) Appl. No.: 19/265,291

(22) Filed: Jul. 10, 2025

Related U.S. Application Data
(60) Provisional application No. 63/673,612, filed on Jul. 19, 2024.

(51) Int. Cl. A61K 8/64; A61K 8/60; A61K 8/98; A61K 8/99; A61K 31/194; A61Q 19/00

(52) U.S. Cl. CPC A61K 8/64; A61K 8/606; A61K 8/987; A61K 8/99; A61K 31/194; A61Q 19/00

(57) ABSTRACT

Polypeptide cosmetic compositions may be useful for addressing skin conditions. Such skin conditions include loss of firmness, lines and wrinkles, and other age-related skin changes associated with the general degradation of the skin dermis, which are often characterized by collagen loss and structural disorganization. Cosmetic compositions may comprise a polypeptide, a first polypeptide activator comprising a guanine nucleotide, and a second polypeptide activator comprising succinic acid. Preferably, the polypeptide has about 5 amino acid residues or less, and more preferably, the polypeptide comprises a tripeptide having an amino acid sequence of Ser-Thr-Pro-NH₂. Preferred polypeptides may promote stimulation of collagen production in skin cells to which the cosmetic compositions are applied.

### FIELD

The present disclosure generally relates to cosmetic compositions, and more specifically, to polypeptide cosmetic compositions that may address skin conditions including skin aging and appearance.

### INCORPORATION BY REFERENCE STATEMENT

An XML sequence listing (filename 2401_084296_000012_1.xml: created Jul. 16, 2025: size=2355 bytes) is submitted herewith and is incorporated by reference in its entirety.

### BACKGROUND

The skin is a vital organ composed of several layers (dermis, epidermal layers, and stratum corneum), which covers the entire surface of the body and serves protective, sensitivity, immune, metabolic, and thermoregulatory functions. The skin, like other organs, is subject to aging.

Intrinsic or chronological aging of the skin and other organs is the consequence of a genetically programmed senescence and biochemical alterations due to endogenous factors. In the skin, the aging process is characterized by a slowdown in the regeneration of cells and extracellular matrices, gradual loss of dermal collagen and elastic fibers that eventually result in dermal and epidermal atrophy, dryness, reduction in elasticity and firmness, the appearance of fine lines and wrinkles, hyperpigmentation or hypopigmentation blemishes, age spots, and the like.

Extrinsic aging of the skin, on the other hand, may occur due to environmental factors such as pollution, sunlight irradiation and other UV irradiation sources, diseases, lifestyle, and the like. Extrinsic and intrinsic aging occur in concert with one another and impact the overall rate at which skin aging occurs.

Many consumers are concerned with the appearance of their skin and the signs of skin aging, such as those noted above. A variety of cosmetic compositions are available to slow the process of skin aging or to protect against the process of skin aging.

Collagen plays a vital role in the extracellular matrix, and is notable as the most prevalent protein in the human body, particularly within the skin. Collagen may be found in fibrillar or non-fibrillar form, both intracellularly and extracellularly. The fibrillar form of collagen is most common and includes collagen subtypes I, II, III, V, and XI, with subtypes I, IV, and V being most often associated with skin and dermal tissue. In its fibrous form, collagen acts as a structural support material for the skin and the external structure of cells, boasting tensile strength, which sets it apart from many other types of proteins. Collagen gives form, firmness, and strength to the skin. However, collagen production dwindles with age, ultimately resulting in a change in skin appearance, such as the development of wrinkles. Consequently, collagen holds a pivotal place for those seeking to combat the visible signs of skin aging.

As such, cosmetic compositions targeting improved production of collagen may be desirable for addressing the age-related impacts of collagen deficiency.

### SUMMARY

In some embodiments, the present disclosure provides cosmetic compositions comprising: a polypeptide that promotes collagen production and comprises about 5 amino acid residues or less: a first polypeptide activator comprising a guanine nucleotide; and a second polypeptide activator comprising succinic acid.

In some or other embodiments, the present disclosure provides cosmetic compositions comprising: a polypeptide having an amino acid sequence of Ser-Thr-Pro-NH₂, the polypeptide being present at about 2.5×10⁻⁵ wt % to about 1.25×10⁻⁴ wt %, based on total mass of the cosmetic composition: a first polypeptide activator comprising a guanine nucleotide, the guanine nucleotide being present at about 1×10⁻³ wt % to about 3.5×10⁻³ wt %, based on total mass of the cosmetic composition; and a second polypeptide activator comprising succinic acid, the succinic acid being present at about 5×10⁻⁵ wt % to about 1.5×10⁻⁴ wt %, based on total mass of the cosmetic composition.

In still other embodiments, the present disclosure provides cosmetic compositions comprising: a polypeptide having an amino acid sequence of Ser-Thr-Pro-NH₂, the polypeptide being present at about 2.5×10⁻⁵ wt % to about 1.25×10⁻⁴ wt %, based on total mass of the cosmetic composition; at least one artemia extract comprising a guanine nucleotide, the guanine nucleotide being present at about 1×10⁻³ wt % to about 3.5×10⁻³ wt %, based on total mass of the cosmetic composition; and at least one extract of Iodobacter ssp. comprising succinic acid, the succinic acid being present at about 5×10⁻⁵ wt % to about 1.5×10⁻⁴ wt %, based on total mass of the cosmetic composition.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a bar graph showing the amount of collagen production promoted by Tripeptide-32 and the treatment composition of the examples.

FIG. 2 is a bar graph showing the amount of collagen production promoted by the treatment composition of the examples, individual components of the treatment composition, and selected mixtures of components of the treatment composition.

FIG. 3 is a bar graph showing the amount of collagen production promoted by the treatment composition of the examples, individual components of the treatment composition, and mixtures of each component of the treatment composition.

### DETAILED DESCRIPTION

The present disclosure generally relates to cosmetic compositions, and more specifically, to polypeptide cosmetic compositions that may address skin conditions including skin aging and appearance.

As discussed above, cosmetic compositions that may improve the appearance of the skin and slow or limit the process of skin aging may be highly desirable for consumers. Stimulation of collagen production may be desirable due to the impact of this biomaterial in the skin aging process.

The present disclosure provides cosmetic compositions that may address the foregoing needs and provide related advantages as well. In particular, the present disclosure provides cosmetic compositions in which a polypeptide containing about 5 amino acid residues or less may interact with at least a first polypeptide activator and a second polypeptide activator to stimulate production of collagen. The first polypeptide activator may comprise a guanine nucleotide, such as diguanosine tetraphosphate, and the second polypeptide activator may comprise succinic acid. Surprisingly, the first and second polypeptide activators in combination may interact with the polypeptide in a manner such that a synergistic increase in collagen production occurs relative to that produced by the polypeptide alone (i.e., when an identical or similar amount of polypeptide is present alone). By a “synergistic interaction,” it is meant that the polypeptide and the first and second polypeptide activators work cooperatively to afford an increase in collagen production that is greater than any one or two components of the cosmetic compositions operating alone at an identical or similar concentration. That is, the increase in collagen production is more than the sum of the contributions for each of the first and second polypeptide activators. Advantageously, the synergistic interaction may be realized within conventional concentration ranges for the individual components of the cosmetic compositions disclosed herein, as discussed hereinafter.

The polypeptide included in the cosmetic compositions disclosed herein may contain 5 amino acid residues or less, such as 3, 4, or 5 amino acid residues, preferably 3 or 4 amino acid residues, and most preferably, 3 amino acid residues (a tripeptide). Suitable tripeptides may already be active to promote collagen production by themselves and exhibit increased collagen production when interacted synergistically with the first and second polypeptide activators according to the description herein. Preferably, the polypeptide may be a listed INCI (International Nomenclature Cosmetic Ingredient).

In specific examples, the polypeptide may have an amino acid sequence of Ser-Thr-Pro-NH₂, in which the C-terminal carboxyl group is replaced with a carboxamide. This polypeptide is more commonly known by its INCI designation of Tripeptide-32. Tripeptide-32 is known to promote collagen production alone when applied topically to the skin. Another suitable polypeptide for stimulating collagen production may be a tetrapeptide having SEQ ID. NO. 1 (Ser-Pro-Leu-Gln-NH₂), which is also known by its INCI designation of Tetrapeptide-26. The C-terminal carboxyl group in Tetrapeptide-26 is likewise replaced with a carboxamide. Tripeptide-32 and Tetrapeptide-26 alone are known to mildly stimulate collagen production. Compositions comprising Tripeptide-32 and Tetrapeptide-26 that may stimulate production of collagen are described in U.S. Pat. No. 10,149,811, for example, which is incorporated herein by reference. In addition to their ability to promote collagen production. Tripeptide-32 and Tetrapeptide-26 are believed to stimulate Circadian Locomotor Output Cycles Kaput (CLOCK) genes and Period Homolog 1 (PER1) in keratinocytes. Preferably, the polypeptide used herein is Tripeptide-32.

Surprisingly, the cosmetic compositions of the present disclosure may exhibit synergistic enhancement of collagen production relative to the polypeptide alone at the same or substantially the same polypeptide concentration. For example, when Tripeptide-32 is combined with the first polypeptide activator alone (a composition containing a guanine nucleotide) or the second polypeptide activator alone (a composition containing succinic acid), little or no enhancement of the production of collagen may occur, relative to that occurring with Tripeptide-32 alone at the same or substantially the same polypeptide concentration. In contrast, when Tripeptide-32 is combined with both the first polypeptide activator and the second polypeptide activator in suitable amounts, significantly increased collagen production may be realized. The increased collagen production when both the first and second polypeptide activators are present in combination with the polypeptide is indicative of their synergistic interaction with the polypeptide.

Accordingly, cosmetic compositions of the present disclosure may comprise: a polypeptide that promotes collagen production and comprises about 5 amino acid residues or less: a first polypeptide activator comprising a guanine nucleotide; and a second polypeptide activator comprising succinic acid. The first polypeptide activator and the second polypeptide activator may interact with the polypeptide to produce a synergistic increase in collagen production relative to the polypeptide alone.

The polypeptide may be present in the cosmetic compositions in an amount ranging from about a1 to about a2 wt %, based on total mass of the cosmetic composition, wherein a1<a2 and a1 and a2 are independently chosen from 2.5×10⁻⁶, 5×10⁻⁶, 7.5×10⁻⁶, 1×10⁻⁵, 1.25×10⁻⁵, 1.5×10⁻⁵, 1.75×10⁻⁵, 2×10⁻⁵, 2.25×10⁻⁵, 2.5×10⁻⁵, 3.75×10⁻⁵, 5×10⁵, 6.25×10⁻⁵, 7.5×10⁻⁵, 8.75×10⁻⁵, 1×10⁻⁴, 1.125×10⁻⁴, or 1.25×10⁻⁴. In non-limiting examples, the polypeptide may be present in the cosmetic compositions in an amount ranging from about 2.5×10⁻⁵ wt % to about 1.25×10⁻⁴ wt %, or about 3.75×10⁻⁵ wt % to about 7.5×10⁻⁵ wt %, or about 4.5×10⁻⁵ wt % to about 6×10⁻⁵ wt %, based on total mass of the cosmetic composition. In some examples, the polypeptide may be present in an amount of about 5×10⁻⁵ wt %, based on total mass of the cosmetic composition. Tripeptide-32 may be present in any of the foregoing wt % ranges.

The first polypeptide activator may comprise a nucleotide, preferably a guanine nucleotide. The term “guanine nucleotide” refers to a compound containing a guanine nucleobase, a sugar (ribose or deoxyribose), and a monomeric phosphate group or a polyphosphate group. The guanine nucleotide may comprise a diguanosine nucleoside, in which two guanine nucleobases are linked together through the monomeric phosphate group or the polyphosphate group. In some examples, the guanine nucleotide may comprise diguanosine tetraphosphate (DGTP) (CAS No. 4130-19-2, also known as diguanosine tetraphosphate).

In some examples, the guanine nucleotide may be present in at least one artemia extract, such as Artemia salina extract. The amount of artemia extract that is used may provide the guanine nucleotide in the amounts referenced below.

The second polypeptide activator may comprise succinic acid, folic acid, or any combination thereof. Preferably, the second polypeptide activator comprises succinic acid.

In some examples, the succinic acid may be present in at least one extract of Iodobacter ssp. The amount of Iodobacter ssp. extract that is used may provide the succinic acid in the amounts referenced below.

The first and second polypeptide activators may be present in suitable amounts and/or in suitable ratios with respect to one another to promote a synergistic interaction with the polypeptide and to stimulate production of collagen to a desired degree.

The first polypeptide activator may be present in the cosmetic compositions in an amount such that the guanine nucleotide is present in an amount ranging from about b1 to about b2 wt %, based on total mass of the cosmetic composition, wherein b1<b2 and b1 and b2 are independently chosen from 1×10⁻³, 1.25×10⁻³, 1.5×10⁻³, 1.75×10⁻³, 2×10⁻³, 2.25×10⁻³, 2.5×10⁻³, 2.75×10⁻³, 3×10⁻³, 3.25×10⁻³, or 3.5×10⁻³. In non-limiting examples, the guanine nucleotide may be present in the cosmetic compositions in an amount ranging from about 1×10⁻³ wt % to about 3.5×10⁻³ wt %, or about 1.5×10⁻³ wt % to about 3×10⁻³ wt %, or about 2×10⁻³ wt % to about 3×10⁻³ wt %, based on total mass of the cosmetic composition. In some examples, the guanine nucleotide may be present in an amount of about 2.5×10⁻³ wt %, based on total mass of the cosmetic composition. Diguanosine tetraphosphate may be present in any of the foregoing wt % ranges.

The second polypeptide activator may be present in the cosmetic compositions in an amount such that succinic acid is present in an amount ranging from about c1 to about c2 wt %, based on total mass of the cosmetic composition, wherein c1<c2 and c1 and c2 are independently chosen from 5×10⁻⁵, 5.5×10⁻⁵, 6×10⁻⁵, 6.5×10⁻⁵, 7×10⁻⁵, 7.5×10⁵, 8×10⁻⁵, 8.5×10⁻⁵, 9×10⁻⁵, 9.5×10⁻⁵, 1.0×10⁻⁴, 1.05×10⁻⁴, 1.1×10⁻⁴, 1.15×10⁻⁴, 1.2×10⁻⁴, 1.25×10⁻⁴, 1.3×10⁻⁴. 1.35×10⁻⁴, 1.4×10⁻⁴, 1.45×10⁻⁴, or 1.5×10⁻⁴. In non-limiting examples, the succinic acid may be present in the cosmetic compositions in an amount ranging from about 5×10⁻⁵ wt % to about 1.5×10⁻⁴ wt %, or about 7×10⁻⁵ wt % to about 1.3×10⁻⁴ wt %, or about 9×10⁻⁵ wt % to about 1.1×10⁻⁴ wt %, based on total mass of the cosmetic composition. In some examples, the succinic acid may be present in an amount of about 1×10⁻⁴ wt %, based on total mass of the cosmetic composition.

In more particular examples, the cosmetic compositions may comprise the polypeptide (e.g., Tripeptide-32) at about 2.5×10⁻⁵ wt % to about 1.25×10⁻⁴ wt %, the succinic acid at about 5×10⁻⁵ wt % to about 1.5×10⁻⁴ wt %, and the guanine nucleotide (e.g., diguanosine tetraphosphate) at about 1×10⁻³ wt % to about 3.5×10⁻³ wt %, each based on total mass of the cosmetic composition. In still more particular examples, the cosmetic compositions may contain the polypeptide (e.g., Tripeptide-32) at about 5×10⁻⁵ wt %, and/or the succinic acid at about 1×10⁻⁴ wt %, and/or the guanine nucleotide (e.g., diguanosine tetraphosphate) at about 2.5×10⁻³ wt %, each based on total mass of the cosmetic composition.

In some or other more particular examples, the cosmetic compositions may comprise the polypeptide (e.g., Tripeptide-32 having an amino acid sequence of Ser-Thr-Pro-NH₂), the polypeptide present at about 2.5×10⁻⁵ wt % to about 1.25×10⁻⁴ wt %, based on total mass of the cosmetic composition; at least one artemia extract comprising a guanine nucleotide, the guanine nucleotide present at about 1×10⁻³ wt % to about 3.5×10⁻³ wt %, based on total mass of the cosmetic composition; and at least one extract of Iodobacter ssp. comprising succinic acid, the succinic acid present at about 5×10⁻⁵ wt % to about 1.5×10⁻⁴ wt %, based on total mass of the cosmetic composition.

The polypeptide (e.g., Tripeptide-32) and the guanine nucleotide (e.g., diguanosine tetraphosphate) may be present in various ratios (wt:wt) with respect to one another. In non-limiting examples, the wt:wt ratio of the polypeptide to the guanine nucleotide may range from about 1:8 to about 1:1400, or about 1:300 to about 1:10, or about 1:30 to about 1:100, or about 1:30 to about 1:60. In some examples, the wt:wt ratio may be about 1:50.

The polypeptide (e.g., Tripeptide-32) and the succinic acid may be present in various ratios (wt:wt) with respect to one another. In non-limiting examples, the wt:wt ratio of the polypeptide to the succinic acid may range from about 2:1 to about 1:60, or about 1:1 to about 1:5, or about 1:1 to about 1:3. In some examples, the wt:wt ratio may be about 1:2.

The guanine nucleotide (e.g., diguanosine tetraphosphate) and the succinic acid may be present in various ratios (wt:wt) with respect to one another. In non-limiting examples, the wt:wt ratio of the guanine nucleotide (e.g., diguanosine tetraphosphate) to the succinic acid may range from about 6.7:1 to about 70:1, or about 50:1 to about 20:1. In some examples, the wt:wt ratio may be about 25:1.

The cosmetic compositions of the present disclosure invention may be in any liquid, semi-solid, or solid form, wherein suitable liquid forms may be an emulsion, solution, or suspension, such as an aqueous emulsion or solution. Suitable liquid forms may comprise about 5 wt % to about 99.9 wt % water, based on total mass of the cosmetic compositions.

It is to be appreciated that additional ingredients that do not interfere with ability of the polypeptide to promote collagen production and/or the synergistic interaction between the polypeptide and the first and second polypeptide activators may also be present in the cosmetic compositions disclosed herein. Non-limiting examples of such additional ingredients may include, for example, preservatives, antioxidants, pH adjusters, solvents, oils, humectants, surfactants, vitamins, antioxidants, fragrances, minerals, particulates, and inert ingredients such as silicones. Examples of such additional ingredients and suitable amounts thereof to include will be familiar to persons having ordinary skill in the art of formulating cosmetic compositions.

Accordingly, the present disclosure provides cosmetic compositions comprising: polypeptide means for promoting collagen production; and first and second polypeptide activator means that collectively interact with the polypeptide means to produce a synergistic increase in collagen production relative to the polypeptide means alone.

The cosmetic compositions of the present disclosure may be applied topically to the skin as part of a user's skincare routine. Optionally, the user may have previously determined that they have a deficiency in collagen synthesis or suspect that they have such a deficiency. The cosmetic compositions may be applied to the skin in a desired location one or more times a day, such in the morning and in the evening after cleansing the skin.

Embodiments disclosed herein include the following:

Embodiment 1. A cosmetic composition comprising:
a polypeptide that promotes collagen production and comprises about 5 amino acid residues or less;
a first polypeptide activator comprising a guanine nucleotide; and
a second polypeptide activator comprising succinic acid.

Embodiment 2. The cosmetic composition of Embodiment 1, wherein the polypeptide is a tripeptide.

Embodiment 3. The cosmetic composition of Embodiment 2, wherein the polypeptide has an amino acid sequence of Ser-Thr-Pro-NH₂.

Embodiment 4. The cosmetic composition of any one of Embodiments 1-3, wherein the polypeptide is present at about 2.5×10⁻⁵ wt % to about 1.25×10⁻⁴ wt %, based on total mass of the cosmetic composition.

Embodiment 5. The cosmetic composition of any one of Embodiments 1-4, wherein the succinic acid is present at about 5×10⁻⁵ wt % to about 1.5×10⁻⁴ wt %, based on total mass of the cosmetic composition.

Embodiment 6. The cosmetic composition of any one of Embodiments 1-5, wherein the guanine nucleotide comprises a diguanosine nucleotide.

Embodiment 7. The cosmetic composition of any one of Embodiments 1-6, wherein the guanine nucleotide comprises diguanosine tetraphosphate.

Embodiment 8. The cosmetic composition of any one of Embodiments 1-7, wherein the guanine nucleotide is present at about 1×10⁻³ wt % to about 3.5×10⁻³ wt %, based on total mass of the cosmetic composition.

Embodiment 9. The cosmetic composition of any one of Embodiments 1-8, wherein the polypeptide is present at about 2.5×10⁻⁵ wt % to about 1.25×10⁻⁴ wt %, the succinic acid is present at about 5×10⁻⁵ wt % to about 1.5×10⁻⁴ wt %, and the guanine nucleotide is present at about 1×10⁻³ wt % to about 3.5×10⁻³ wt %, each based on total mass of the cosmetic composition.

Embodiment 10. The cosmetic composition of any one of Embodiments 1-8, wherein the polypeptide is present at about 5×10⁻⁵ wt %, based on total mass of the cosmetic composition.

Embodiment 11. The cosmetic composition of any one of Embodiments 1-10, wherein the polypeptide is present at about 5×10⁻⁵ wt %, based on total mass of the cosmetic composition.

Embodiment 12. The cosmetic composition of any one of Embodiments 1-10, wherein the succinic acid is present at about 1×10⁻⁴ wt %, based on total mass of the cosmetic composition.

Embodiment 13. The cosmetic composition of any one of Embodiments 1-10, wherein the guanine nucleotide is present at about 2.5×10⁻³ wt %, based on total mass of the cosmetic composition.

Embodiment 14. The cosmetic composition of any one of Embodiments 1-13, wherein the guanine nucleotide is present in at least one artemia extract.

Embodiment 15. The cosmetic composition of Embodiment 14, wherein the at least one artemia extract comprises at least Artemia salina extract.

Embodiment 16. The cosmetic composition of any one of Embodiments 1-15, wherein the succinic acid is present in at least one extract of Iodobacter ssp.

Embodiment 17. A cosmetic composition comprising:
a polypeptide having an amino acid sequence of Ser-Thr-Pro-NH₂, the polypeptide being present at about 2.5×10⁻⁵ wt % to about 1.25×10⁻⁴ wt %, based on total mass of the cosmetic composition;
a first polypeptide activator comprising a guanine nucleotide, the guanine nucleotide being present at about 1×10⁻³ wt % to about 3.5×10⁻³ wt %, based on total mass of the cosmetic composition; and
a second polypeptide activator comprising succinic acid, the succinic acid being present at about 5×10⁻⁵ wt % to about 1.5×10⁻⁴ wt %, based on total mass of the cosmetic composition.

Embodiment 18. The composition of Embodiment 17, wherein the first polypeptide activator and the second polypeptide activator interact with the polypeptide to produce a synergistic increase in collagen production relative to the polypeptide alone.

Embodiment 19. The cosmetic composition of Embodiment 17 or Embodiment 18, wherein the guanine nucleotide comprises diguanosine tetraphosphate.

Embodiment 20. A cosmetic composition comprising:
a polypeptide having an amino acid sequence of Ser-Thr-Pro-NH₂, the polypeptide being present at about 2.5×10⁻⁵ wt % to about 1.25×10⁻⁴ wt %, based on total mass of the cosmetic composition;
at least one artemia extract comprising a guanine nucleotide, the guanine nucleotide being present at about 1×10⁻³ wt % to about 3.5×10⁻³ wt %, based on total mass of the cosmetic composition; and
at least one extract of Iodobacter ssp. comprising succinic acid, the succinic acid being present at about 5× 10⁻⁵ wt % to about 1.5×10⁻⁴ wt %, based on total mass of the cosmetic composition.

Embodiment 21. A method comprising:
applying the cosmetic composition of any one of Embodiments 1-20 to a skin surface; and
stimulating collagen production within the skin surface.

To facilitate a better understanding of the present disclosure, the following examples of preferred or representative embodiments are given. In no way should the following examples be read to limit, or to define, the scope of the invention.

### EXAMPLES

Treatment Composition: A treatment composition was formulated as an aqueous solution containing about 5×10⁻⁵ wt %, of a tripeptide having a sequence of Ser-Thr-Pro-NH₂ (INCI designation, Tripeptide-32), about 1× 10⁻⁴ wt %, of succinic acid, and about 2.5×10⁻³ wt %, of diguanosine tetraphosphate, based on the total weight of the treatment composition. The succinic acid was obtained as an extract of Iodobact ssp. spray coated onto maltodextrin, which was then dissolved in water. The diguanosine tetraphosphate (DGTP) was obtained as an extract of Artemia salina.

Seeding Cells: Normal human dermal fibroblasts (NHDFs) from two donors (61 and 62 years of age) were obtained and plated onto one 48-well plate at a density of 50.000 cells/well and incubated at standard conditions (37° C./5% CO₂) overnight. NHDFs were grown in DMEM media supplemented with 10% bovine calf serum.

Preparation & Treatment: Following incubation, media was aspirated from the wells and replaced with either fresh media, a reference compound (positive control), the treatment composition described above, individual components of the treatment composition, or mixtures of the components of the treatment composition. After loading, the 48-well plate was then incubated for 72 hours at standard conditions (37° C. and 5% CO₂).

Cell Counts: After a 72 hours incubation and supernatant collection, cell counts were performed for cell viability. Cell counts were conducted with a Cellaca high-throughput cell counting machine.

Collagen ELISA: Collagen I production was assessed using the Pro Collagen Type I ELISA (Takara MK101) per vendor's protocol (Takara Bio. MK101). The optical density (OD) was measured at 450 nm.

Statistical Analysis: Statistical analysis was performed on all samples using one way ANOVA with Dunnett's post hoc in GraphPad Prism 9. Error bars were calculated from the raw data by Microsoft Excel.

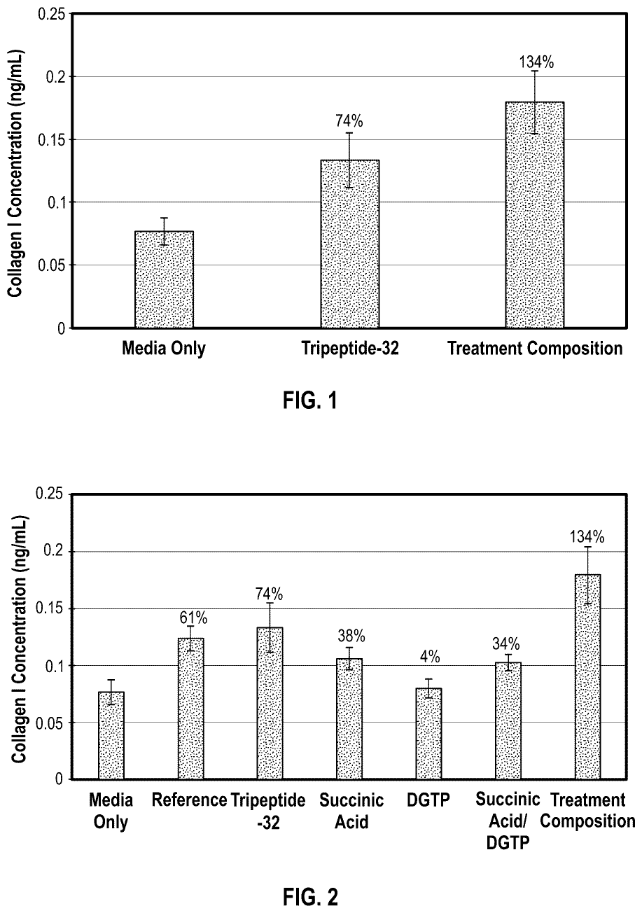

Results: FIG. 1 is a bar graph showing the amount of collagen production promoted by Tripeptide-32 and the treatment composition, in comparison to a blank media control. The results in FIG. 1 are averaged for the 61-year and 62-year old subjects and are normalized to the average cell count. The percentage change for Tripeptide-32 and the treatment composition in FIG. 1 are calculated relative to the blank media control. As shown. Tripeptide-32 alone increased collagen production by 74% relative to the control, whereas the treatment composition more than doubled collagen production at 134% relative to the control. Thus. Tripeptide-32 afforded more collagen production in the presence of the succinic acid and DGTP than did an equal quantity of Tripeptide-32 alone

FIG. 2 is a bar graph showing the amount of collagen production promoted by the treatment composition, individual components of the treatment composition, and selected mixtures of components of the treatment composition, in comparison to a blank media control and a L-ascorbic acid reference (18 μg/mL, positive control). Individual components or mixtures of components tested in FIG. 2 were formulated at the same concentrations present in the treatment composition. The results in FIG. 2 are averaged for the 61-year and 62-year old subjects and are normalized to the average cell count. The percentage change values in FIG. 2 are calculated relative to the blank media control. As shown, the treatment composition again more than doubled collagen production relative to the control (134% increase), whereas Tripeptide-32 alone at the same concentration produced a much more modest increase of 74%.

Referring still to FIG. 2, the individual components of the treatment composition afforded less collagen production than did Tripeptide-32. Succinic acid alone afforded approximately half the increase in collagen production afforded by Tripeptide-32, and DGTP afforded essentially the same amount of collagen production as did the control. Moreover, combining DGTP with succinic acid afforded essentially the same amount of collagen production as did succinic acid alone. This result suggests that succinic acid and DGTP may afford increased collagen production through a synergistic interaction with Tripeptide-32.

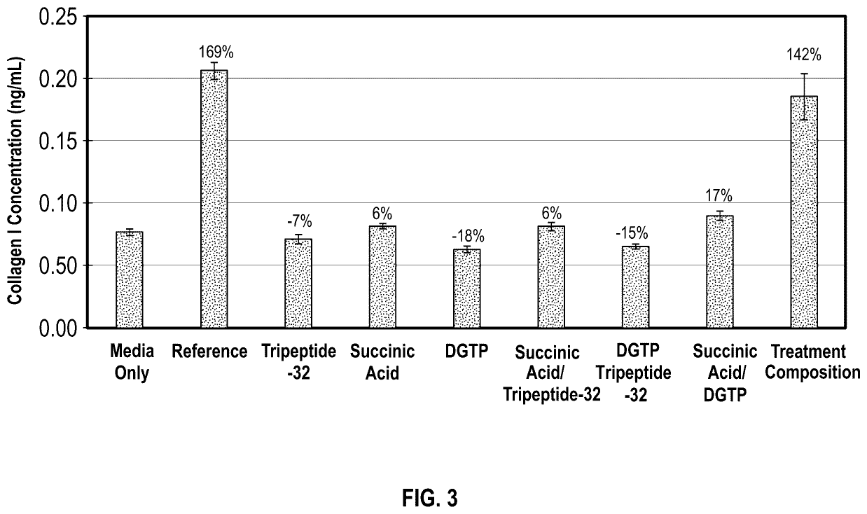

The possible synergistic interaction of succinic acid and DGTP was further investigated by testing additional combinations of the components of the treatment composition for their impact on collagen production. FIG. 3 is a bar graph showing the amount of collagen production promoted by the treatment composition, individual components of the treatment composition, and mixtures of each component of the treatment composition, in comparison to a blank media control and a L-ascorbic acid reference (18 μg/mL, positive control). Individual components or mixtures of components in FIG. 3 were formulated at the same concentrations present in the treatment composition. The results in FIG. 3 were obtained using only the NHDFs obtained from the 62-year old subject and are again normalized to the average cell count. The percentage change values in FIG. 3 are calculated relative to the blank media control. In this case, the individual components of the treatment composition exhibited minimally increased (or even decreased) collagen production relative to the control, and binary combinations of the individual components of the treatment composition likewise afforded minimally increased (or even decreased) collagen production relative to the control. In contrast, the treatment composition containing all components in combination with one another afforded a robust increase of 142% relative to the control. The results in FIG. 3 demonstrate that the combination of Tripeptide-32, succinic acid, and DGTP affords a synergistic effect with respect to collagen production, since none of the components alone or in binary combinations with one another produce a similar increase in collagen production.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including,” Whenever a method, composition, element, or group of elements is preceded with the transitional phrase “comprising.” it is understood that we also contemplate the same composition or group of elements with transitional phrases “consisting essentially of,” “consisting of,” “selected from the group consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently. “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40) mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is the following:

1. A cosmetic composition comprising:
a polypeptide that promotes collagen production and comprises about 5 amino acid residues or less;
a first polypeptide activator comprising a guanine nucleotide; and
a second polypeptide activator comprising succinic acid.

2. The cosmetic composition of claim 1, wherein the polypeptide is a tripeptide.

3. The cosmetic composition of claim 2, wherein the polypeptide has an amino acid sequence of Ser-Thr-Pro-NH₂.

4. The cosmetic composition of claim 1, wherein the polypeptide is present at about 2.5×10⁻⁵ wt % to about 1.25×10⁻⁴ wt %, based on total mass of the cosmetic composition.

5. The cosmetic composition of claim 1, wherein the succinic acid is present at about 5×10⁻⁵ wt % to about 1.5×10⁻⁴ wt %, based on total mass of the cosmetic composition.

6. The cosmetic composition of claim 1, wherein the guanine nucleotide comprises a diguanosine nucleotide.

7. The cosmetic composition of claim 6, wherein the guanine nucleotide comprises diguanosine tetraphosphate.

8. The cosmetic composition of claim 1, wherein the guanine nucleotide is present at about 1×10⁻³ wt % to about 3.5×10⁻³ wt %, based on total mass of the cosmetic composition.

9. The cosmetic composition of claim 1, wherein the polypeptide is present at about 2.5×10⁻⁵ wt % to about 1.25×10⁻⁴ wt %, the succinic acid is present at about 5×10⁻⁵ wt % to about 1.5×10⁻⁴ wt %, and the guanine nucleotide is present at about 1×10⁻³ wt % to about 3.5×10⁻³ wt %, each based on total mass of the cosmetic composition.

10. The cosmetic composition of claim 9, wherein the polypeptide is present at about 5×10⁻⁵ wt %, based on total mass of the cosmetic composition.

11. The cosmetic composition of claim 9, wherein the succinic acid is present at about 1×10⁻⁴ wt %, based on total mass of the cosmetic composition.

12. The cosmetic composition of claim 9, wherein the guanine nucleotide is present at about 2.5×10⁻³ wt %, based on total mass of the cosmetic composition.

13. The cosmetic composition of claim 1, wherein the guanine nucleotide is present in at least one artemia extract.

14. The cosmetic composition of claim 13, wherein the at least one artemia extract comprises at least Artemia salina extract.

15. The cosmetic composition of claim 1, wherein the succinic acid is present in at least one extract of Iodobacter ssp.

16. A cosmetic composition comprising:
a polypeptide having an amino acid sequence of Ser-Thr-Pro-NH₂, the polypeptide being present at about 2.5×10⁻⁵ wt % to about 1.25×10⁻⁴ wt %, based on total mass of the cosmetic composition;
a first polypeptide activator comprising a guanine nucleotide, the guanine nucleotide being present at about 1×10⁻³ wt % to about 3.5×10⁻³ wt %, based on total mass of the cosmetic composition; and
a second polypeptide activator comprising succinic acid, the succinic acid being present at about 5×10⁻⁵ wt % to about 1.5×10⁻⁴ wt %, based on total mass of the cosmetic composition.

17. The composition of claim 16, wherein the first polypeptide activator and the second polypeptide activator interact with the polypeptide to produce a synergistic increase in collagen production relative to the polypeptide alone.

18. The cosmetic composition of claim 16, wherein the guanine nucleotide comprises diguanosine tetraphosphate.

19. A cosmetic composition comprising:
a polypeptide having an amino acid sequence of Ser-Thr-Pro-NH₂, the polypeptide being present at about 2.5×10⁻⁵ wt % to about 1.25×10⁻⁴ wt %, based on total mass of the cosmetic composition;
at least one artemia extract comprising a guanine nucleotide, the guanine nucleotide being present at about 1×10⁻³ wt % to about 3.5×10⁻³ wt %, based on total mass of the cosmetic composition; and
at least one extract of Iodobacter ssp. comprising succinic acid, the succinic acid being present at about 5×10⁻⁵ wt % to about 1.5×10⁻⁴ wt %, based on total mass of the cosmetic composition.

20. A method comprising:
applying the cosmetic composition of claim 1 to a skin surface; and
stimulating collagen production within the skin surface.


SEQUENCE LISTING (1 sequence)

SEQ ID NO: 1 - protein, 4 aa, Synthetic construct
  MOD_RES 4: note = The C-terminal carboxyl group of Gln is replaced with a carboxamide.
SPLQ